(19) United States
(12) Patent Application Publication
(10) Pub. No.: US 2007/0232567 A1
(43) Pub. Date: Oct. 4, 2007

(54) Formulations Of Low Dose Non-Steroidal Anti-Inflammatory Drugs And Beta-Cyclodextrin

(76) Inventors: Curtis Wright, Rockport, MA (US); Daniel B. Carr, Chestnut Hill, MA (US); Fred H. Mermelstein, Newton, MA (US)

Correspondence Address: BAKER BOTTS L.L.P., 30 ROCKEFELLER PLAZA, 44th Floor, NEW YORK, NY, 10112-4498, US

(21) Appl. No.: 11/689,939

(22) Filed: Mar. 22, 2007

Related U.S. Application Data
(60) Provisional application No. 60/786,487, filed on Mar. 28, 2006.

(51) Int. Cl. A61K 31/724; A61K 31/415; A61K 31/405; A61K 31/192; A61K 31/365

(52) U.S. Cl. 514/58; 514/406; 514/471; 514/569; 514/570; 514/420

(57) ABSTRACT

The present invention is directed to pharmaceutical compositions containing (a) a dosage of a non-steroidal anti-inflammatory drug (NSAID) effective to induce analgesia an anti-inflammatory effect, or an anti-pyretic effect and (b) a beta-cyclodextrin compound; wherein the dosage of the NSAID compound is less than the minimum approved dose for the route of administration. Additionally, the present invention is directed to methods for treating a mammal in need of an analgesic, an anti-inflammatory, or an anti-pyretic agent comprising administering the pharmaceutical composition of the present invention.

### CROSS-REFERENCE TO RELATED APPLICATIONS

This application claims priority under 35 U.S.C. § 119, based on U.S. Provisional Application Ser. No. 60/786,487, filed Mar. 28, 2006, the disclosure of which is incorporated herein by reference in its entirety.

### FIELD OF THE INVENTION

The present invention is directed to pharmaceutical compositions containing NSAIDS in amounts lower than the minimum approved dosage and beta-cyclodextrin compounds. The present invention is also directed methods of treating a subject with the pharmaceutical compositions of the present invention.

### BACKGROUND OF THE INVENTION

Diclofenac is a well-known non-steroidal anti-inflammatory drug (“NSAID”) used in acute and chronic pain in both parenteral and oral dosage forms. Oral dosages range from 100-200 mg/day, while parenteral dosages range from 75-150 mg/day (1-2 mg/kg/day) by either infusion or intermittent (divided) doses. Toxicity of oral and parenteral forms are well known, with gastrointestinal, hemorrhagic, renal, hepatic, cardiovascular and allergic (anaphylactic and severe dermal allergy) adverse events being most significant.

Parenteral use of diclofenac has been limited due to limited solubility, such that parenteral preparations have had to include non-polar solvents in order to achieve concentrations (75 mg/3 ml) which would allow intra-muscular (IM) administration of the desired dose. This solubility has limited the parenteral use to IM use and/or slow intravenous (IV) administration of diluted (100-500 ml diluent) product.

U.S. Pat. No. 5,679,660 and co-pending application Ser. No. 10/999,155, filed Nov. 30, 2004, published as US 2005/0238674 A1 on Oct. 27, 2005, both of which are incorporated by reference, disclose novel formulations of diclofenac with hydroxypropyl-beta-cyclodextrin, which allows a more concentrated preparation and thus rapid intravenous administration. The data show that the more concentrated diclofenac/beta-cyclodextrin formulation shows faster onset of action than current products.

Other than ease of administration and more rapid onset of action, consequent on the improvements in the pharmaceutical formulation, no other advantages were observed. The present invention arises, in part, from the surprising discovery that formulating a non-steroidal anti-inflammatory drug with beta-cyclodextrin not only improves solubility and stability of the drug, it also increases efficacy.

### SUMMARY OF THE INVENTION

In one embodiment, the invention provides a pharmaceutical composition comprising a dosage of a non-steroidal anti-inflammatory drug (NSAID) effective to induce analgesia an anti-inflammatory effect, or an anti-pyretic effect and a beta-cyclodextrin compound. The dosage of the NSAID compound is less than the minimum approved dose for the route of administration. In a specific embodiment, the NSAID is not a diclofenac compound. In a specific embodiments, the dosage of the NSAID is less than about 50%, or less than about 25%, of the minimum approved dose for the route of administration. NSAIDs suitable for use in the invention include those that are solubilized by a beta-cyclodextrin compound, which can readily be determined through routine experimentation. In another embodiment, the NSAIDs are those whose efficacy is improved by formulation with a beta-cyclodextrin compound, which can be determined through routine experimentation. Such NSAIDs may include diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib or lumaricoxib.

In a specific embodiment, the cyclodextrin compound is 2-hydroxypropyl-beta-cyclodextrin.

The invention also provides a method for treating a mammal in need of an analgesic, an anti-inflammatory, or an anti-pyretic agent, which comprises administering a pharmaceutical composition as set forth above. In particular embodiments, the pharmaceutical composition can be administered intramuscularly or intravenously.

In another embodiment, the invention provides a method for treating a mammal in need of an analgesic, an anti-inflammatory, or an anti-pyretic agent, which comprises administering a pharmaceutical composition comprising a dosage of an NSAID effective to induce analgesia an anti-inflammatory effect, or an anti-pyretic effect; and a beta-cyclodextrin compound, in which the dosage of the NSAID is less than the minimum approved dose for the route of administration. In a specific embodiment, the NSAID is not diclofenac. NSAIDs for use in this method are as described above. In a specific embodiment, the dose of the NSAID has the same efficacy as the minimum approved dosage. In another embodiment, the NSAID has from about 70% to about 100% or from about 40% to about 70% of the efficacy of the minimum approved dosage.

In another embodiment, the NSAID has the same duration as the minimum approved dosage. In another embodiment, the dosage of the NSAID has from about two-thirds to the same duration, or one-third to about two-thirds of the duration, as the minimum approved dosage.

### DESCRIPTION OF THE FIGURES

FIG. 1 contains a graphical representation of the 100 mm visual analog pain relief (mm) afforded to patients over time (hours) based on the formulation strengths administered. The tested formulations include placebo, 3.75 mg Dyloject, 9.4 mg Dyloject, 18.75 mg Dyloject; 37.5 mg Dyloject, 75 mg Dyloject, and 30 mg Ketorolac.

FIG. 2 illustrates the dose-response curve for peak analgesia observed (mm VAS) over mg of formulation. Both diclofenac and ketorolac formulations were tested.

FIG. 3 illustrates the dose-duration relationship examined using the median time to re-medication (hours) in the single dose phase. Two formulations of diclofenac were studied.

FIG. 4 illustrates the percentage of patients with NSAID Adverse Events by dose of diclofenac (mg).

### DETAILED DESCRIPTION OF THE INVENTION

The present invention provides formulations of non-steroidal anti-inflammatory drugs (NSAIDs) with a beta-cyclodextrin. These formulations unexpectedly provide for significant efficacy and duration of pain relief at a lower dose of the NSAID. More particularly, at a reduced dose and dosage the formulation provides the same level of efficacy and the same duration of analgesia as the minimum approved dose and dosage.

The invention is based, in part, on results of a comparison of the efficacy of diclofenac solubilized with hydroxypropyl-beta-cyclodextrin to ketorolac and placebo for the treatment of moderate-to-severe post-surgical pain. The efficacy of diclofenac solubilized with hydroxypropyl-β-cyclodextrin at several dose levels suggests a faster onset of action. Most notably, diclofenac formulated with hydroxypropyl-beta-cyclodextrin provides single-dose efficacy at 50%, 25%, 12.5% and 5% of the current recommended doses of diclofenac. This in combination with the known human pharmacokinetic results for the formulation supports reduced total daily doses of this NSAID with anticipated lower risk of toxicity by reducing the extent and duration of drug exposure. This is a novel finding and of clinical importance.

The minimum effective dose of diclofenac solubilized with hydroxypropyl-β-cyclodextrin tested was 3.75 mg, demonstrating that diclofenac, if solubilized with hydroxypropyl-beta-cyclodextrin, may be administered at doses lower than those previously considered necessary for postoperative analgesia. Moreover, because the increased efficacy results from solubilization with hydroxypropyl-beta-cyclodextrin, the results observed with diclofenac, which are independent of the solubility improvement observed previously, demonstrate the ability to increase the efficacy of other NSAIDs, and concomitantly enhance the safety of NSAID analgesia.

While the present invention is based on the results with diclofenac, which demonstrate the ability of administration of an NSAID with a beta-cyclodextrin to increase efficacy of the NSAID, compositions of diclofenac and beta-cyclodextrin and methods of using them are the subject of a separate patent application, entitled FORMULATIONS OF LOW DOSE DICLOFENAC AND BETA-CYCLODEXTRIN, filed on even date herewith, and assigned attorney docket number 077350.0221. Accordingly, in certain aspects the invention relates to formulations of an NSAID and beta-cyclodextrin and uses of such formulations, in which the NSAID is not diclofenac.

The term “NSAID” as used herein includes but is not limited to diclofenac, diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib or lumaricoxib.

The term “diclofenac compound” refers to diclofenac or a pharmaceutically acceptable diclofenac salt. A pharmaceutically acceptable salt of diclofenac, can be an alkali metal salt, for example the sodium or the potassium salt, or the salt formed with an amine, e.g., a mono-, di- or tri-C₁-C₄ alkylamine, for example diethyl- or triethyl-amine, hydroxy-C₂-C₄ alkylamine, for example ethanolamine, or hydroxy-C₂-C₄ alkyl-C₁-C₄ alkylamine, for example dimethylethanolamine, or a quaternary ammonium salt, for example the tetramethylammonium salt or the choline salt of diclofenac (see, e.g., U.S. Pat. No. 5,389,681). Preferably the diclofenac salt is diclofenac sodium.

Suitable formulations of the present invention for parenteral administration include cyclodextrin inclusion complexes. One or more modified or unmodified cyclodextrins can be employed to stabilize and increase the water solubility and efficacy of compounds of the present invention. Useful cyclodextrins for this purpose include beta-cyclodextrins. The term “beta-cyclodextrin” as used herein refers to cyclic alpha-1,4-linked oligosaccharides of a D-glucopyranose containing a relatively hydrophobic central cavity and hydrophilic outer surface. Particular efficacy has been observed in the present invention utilizing hydroxypropyl-beta-cyclodextrin, however, other substituted and unsubstituted beta-cyclodextrins can also be used in the practice of the invention. Additional examples of cyclodextrins that may be utilized are disclosed in U.S. Pat. Nos. 4,727,064, 4,764,604, 5,024,998, 6,407,079, 6,828,299, 6,869,939 and Jambhekar et al., 2004 Int. J. Pharm. 2004, 270(1-2) 149-66. The formulations may be prepared as described in U.S. Pat. Nos. 5,679,660 and 5,674,854.

The “pharmaceutical compositions” for use in accordance with the present invention can be formulated in any conventional manner using one or more pharmaceutically acceptable carriers or excipients. A “pharmaceutically acceptable” carrier or excipient, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

Pharmaceutical compositions include solid dosage forms, e.g., for perioral, transnasal (powder), or rectal (suppository) administration; and liquid dosage forms, e.g., for parenteral administration (injection), transnasal (spray), or perioral administration. In a specific embodiment, the pharmaceutical compositions of the present invention are liquid compositions formulated for intravenous or intramuscular administration, and particularly intravenous administration.

As used herein, the term “stabilizer” refers to a compound optionally used in the pharmaceutical compositions of the present invention in order to avoid the need for sulphite salts and increase storage life. Optimal stabilizers include antioxidants, specifically monothioglycerol and those described in U.S. Patent Publication 2005/0238674.

The term “dosage” is intended to encompass a formulation expressed in terms of mg/kg/day. The dosage is the amount of an ingredient administered in accordance with a particular dosage regimen. A “dose” is an amount of an agent administered to a subject in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning in any case will be apparent from context.

The term “mammal” is intended to include, any warm-blooded vertebrate having the skin more or less covered with hair. Most preferably, the mammal is a human subject, but the mammal can also be a non-human animal. Thus, the invention is useful in veterinary medicine as well, e.g., for treating pain in a domestic pet, such as a canine or feline, a farm animal, a work animal, or an animal in a circus or zoological garden. The invention has particular value in treating pain in a horse, particularly in sport, such as thoroughbred and other race horses, rodeo horses, circus horses, and dressage horses. A particular advantage of the invention is that, by increasing the efficacy of a dosage of the NSAID, it is possible to administer a therapeutic dosage that is below a maximum allowed dose permitted by the particular regulatory authorities of the sport.

The term “minimum approved dose” refers to the minimum dosage that has received full regulatory approval by the appropriate United States or foreign regulatory authority as safe and effective for human or veterinary use.

The term “therapeutically effective” as applied to dose or amount refers to that quantity of a compound or pharmaceutical composition that is sufficient to result in a desired activity upon administration to a mammal in need thereof. As used herein with respect to the pharmaceutical compositions comprising an antifungal, the term “therapeutically effective amount/dose” refers to the amount/dose of a compound or pharmaceutical composition that is sufficient to produce an effective response upon administration to a mammal.

The term “amount” as used herein refers to quantity or to concentration as appropriate to the context. In the present invention, the effective amount of a compound refers to an amount sufficient to treat a patient/subject in need of analgesia. The effective amount of a drug that constitutes a therapeutically effective amount varies according to factors such as the potency of the particular drug, the route of administration of the formulation, and the mechanical system used to administer the formulation. A therapeutically effective amount of a particular drug can be selected by those of ordinary skill in the art with due consideration of such factors.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

### Methods of Treatment

As noted above, the novel dosage formulations of the invention are suitable for administering an NSAID for any purpose, including to treat pain (analgesia), to treat fever (anti-pyretic), and to treat inflammation (anti-inflammatory). Various embodiments of the invention provide for administration of unit doses to achieve a total dosage for the desired effect. The examples demonstrate efficacy of a 3.75 mg dose of diclofenac, which is about 5% of the minimum approved dose (and about 5% or about 2.5% of the approved daily dosage). However, this dose provides about 40% of the pain relief and one-third of the duration as the minimum approved dose. Better results can be achieved by selecting a dosage regiment with this dose of NSAID, e.g., increasing the frequency of administration, to achieve a level and duration of effect acceptable for the patient. Higher dose formulations likewise could provide such an effect. Such higher dose formulations are nevertheless lower than any approved formulation, and the dosage regimen results in administration of less NSAID than the current approved minimum dosage regimen.

A significant advantage of the invention results from the ability to achieve efficacy with lower doses and overall daily dosing of the NSAID. Consequently, it is possible to reduce the dosage, and thus reduce toxicity.

In specific embodiments, the unit dose (i.e., the amount of active drug administered at one time to a patient) is no more than about 75%, no more than about 50%, no more than about 25%, no more than about 12.5%, and no more than about 5%, of the approved minimum dose. Doses that are about or greater than about 50% of the approved minimum dose can show the same level and duration of pain relieve as the minimum effective dose. Furthermore, by increasing the frequency of administration of a lower dose formulation, the patient can achieve the same levels of efficacy and duration of pain relief as with the approved doses, with decreased toxicity.

In another embodiment, then, the invention provides for titrating the dose reduction of the NSAID and beta-cyclodextrin by decreasing the unit dose to achieve an effect (analgesia, anti-inflammatory, and/or anti-pyretic) that is sufficient, even at a reduced level, for the patient's needs, which can be met by increasing the dosing frequency to achieve an effective daily dosage that is still lower than the minimum approved dose. The term “effect” means that there is a statistically significant difference in a response in patients taking the formulation containing the NSAID relative to patients taking a placebo.

The formulations of the invention can be administered via any route, including parenteral, perioral, transnasal, and rectal. Particular parenteral routes of administration include intravenous and intramuscular injection.

The NSAID actives of the formulations of the invention are suitable for treating pain, fever, and inflammation. In particular, the formulations are suitable in the treatment of acute painful conditions in humans and animals such as headache, including migraine, trauma, dysmenorrhoea, renal or biliary colic, post-operative pain, gout, arthritis, cancer related pain, musculoskeletal pain, lower back pain, fibromyalgia, and pain of infectious origin. In a specific embodiment, exemplified below, the formulation is effective to treat post-surgical dental pain resulting from surgical extraction of one or more third molars. In addition, although not intending to be bound by any particular mechanism of action, the formulation of the invention may be used prophylactically to prevent the formation of prostaglandins during and after surgery, with subsequent reduction in immediate post-operative pain. Further, the formulation of the invention may be used to modulate nuclear transcription factors, such as NF-κB, or to modulate ion channel activity, for example as described in Ocana, Maria et al., Potassium Channels and Pain: Present Realities and Future Opportunities, 500 Eur. J. Pharm. 203 (2004).

### EXAMPLES

The present invention will be better understood by reference to the following Examples, which are provided as exemplary of the invention, and not by way of limitation.

### Example 1

### Analysis of Pain Relief Afforded to Patients Based on Administered Dose

A 336-patient, seven treatment arm, randomized, double-blind, single-dose, and placebo- and comparator-controlled, parallel-group study was conducted. Patients were randomly assigned to receive a single dose of either diclofenac sodium solubilized with hydroxypropyl-beta-cyclodextrin (hereinafter “DIC”), ketorolac tromethamine, or placebo.

Bolus IV injectable 2 ml solutions were prepared by solubilizing diclofenac sodium with hydroxypropyl-beta-cyclodextrin. The formulation strengths were as follows:
Formulation: Diclofenac sodium solubilized with hydroxypropyl-β-cyclodextrin
Strengths: 75 mg, 37.5 mg, 18.75 mg, 9.4 mg and 3.75 mg
Dosage: Bolus IV injection (no less than 15 sec)
Batch Number: 063004 (PPS4010)
Manufacturer: Manufactured for Javelin by Precision Pharma
Storage Conditions: Room temperature

Active Control/Comparator:
Formulation: Ketorolac Tromethamine
Strength: 30 mg
Dosage: Bolus IV injection (no less than 15 sec)
Batch Number: 21-430-DK
Manufacturer: Abbott Labs
Storage Conditions: Room temperature

Pain was assessed by each patient at Baseline (0 hour: Visual Analog Scale (VAS) and categorical pain intensity only), at 5, 15, 30 and 45 minutes, and at 1, 1.5, 2, 3, 4, 5, 6, 7, 8, 10, 12 and 24 hours after administration of study medication and immediately prior to the first dose of rescue medication. At each post-dose time period, levels of pain intensity (categorical and VAS) and pain relief (categorical and VAS) was assessed by each patient. Patients were also provided with 2 stopwatches to measure perceptible and meaningful pain relief.

The presence of a dose-response was tested with a step-down testing procedure. Total pain relief (TOTPAR), peak pain relief, pain intensity difference (SPID), summed peak reduction in pain intensity difference (SPRID), and patient global evaluation was analyzed with analysis of variance (ANOVA) with treatment, center, and baseline categorical pain intensity as factors. The possibility of interactions was investigated. Comparisons of the DIC groups with the placebo and Ketorolac groups were performed with Dunnett's test. The presence of a linear dose response for the ordered DIC dose levels was tested with orthogonal contrasts for TOTPAR, SPID and SPRID. Tests of individual DIC dose levels versus placebo for TOTPAR, SPID, and SPRID were conducted with the Tukey, Ciminera, and Heyse step-down testing procedure. The mean, standard deviation, and sample size were presented for each treatment group. Significant p-values (those less than or equal to 0.05) were presented for each step of the procedure. Non-significant p-values were represented with three dashes. Time to onset of perceptible relief and time to onset of meaningful relief was analyzed with survival analysis techniques. These variables were summarized with number of observations, mean, standard deviation, median, and range. Log-rank tests were used to compare treatments with respect to survival distributions. The median time to event for each treatment group was estimated with the Kaplan-Meier product limit estimator. A 95% confidence interval for each estimated median time to event was calculated.

The results of the study were strongly positive, novel and could not have been anticipated from prior experience with diclofenac. The doses had been chosen based on the currently recommended minimally effective doses of 1 mg/kg (50 mg immediate-release or 100 mg sustained-release orally or 75 mg intramuscularly or intravenously). Based on these doses the test conditions were a full dose (75 mg), half dose (37.5) mg, a possibly effective dose (18.75 mg) and two placebo doses (9.75 & 3.4 mg). The findings were as follows:

TABLE 1
TOTPAR (Sum of Pain Relief VAS 0–100 mm ratings 0–6 hours)

| Treatment Group | Result | % Maximum Effect |
|---|---|---|
| Placebo | 62.8 (SEM 9) | 17% |
| DIC 3.75 mg | 134.1 (SEM 8) | 38% |
| DIC 9.4 mg | 237.6 (SEM 15) | 68% |
| DIC 18.75 mg | 284.4 (SEM 21) | 82% |
| DIC 37.5 mg | 348.2 (SEM 30) | 100% |
| DIC 75 mg | 346.3 (SEM 27) | 100% |
| ketorolac | 400.3 (SEM 36) | 100% |

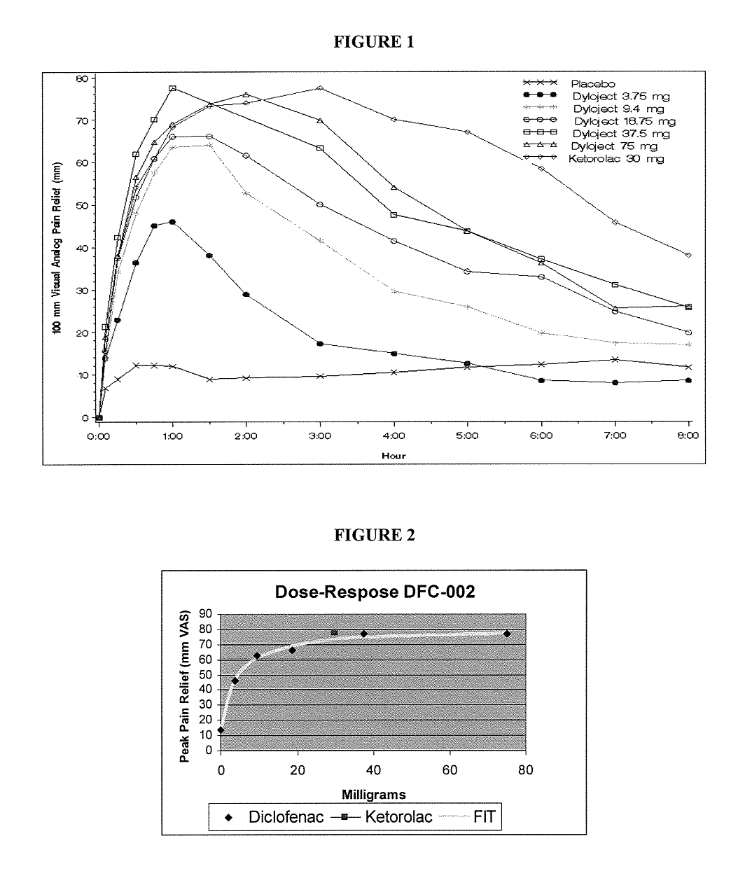

See FIG. 1 for the corresponding graphical representation of the pain relief afforded to patients based on the formulation strengths administered.

### Example 2

### Analysis of Efficacy and Duration of Pain Relief at Lower Doses of Diclofenac

To explore this further, the dose-duration relationship in the same study was examined using the median time to remedication in the single-dose phase. Utilizing the results of study in Example 1, the efficacy and duration of pain relief were thoroughly analyzed. The lowest IV dose of DIC (3.75 mg) had 38% of the effect of the maximal dose, and the next lowest dose (9.4 mg) had 68% of the maximal possible effect, despite being 5% and 12% respectively of the current recommended minimally effective dose (1 mg/kg). FIG. 2 contains a graphical illustration of the dose-response for peak analgesia observed in the study.

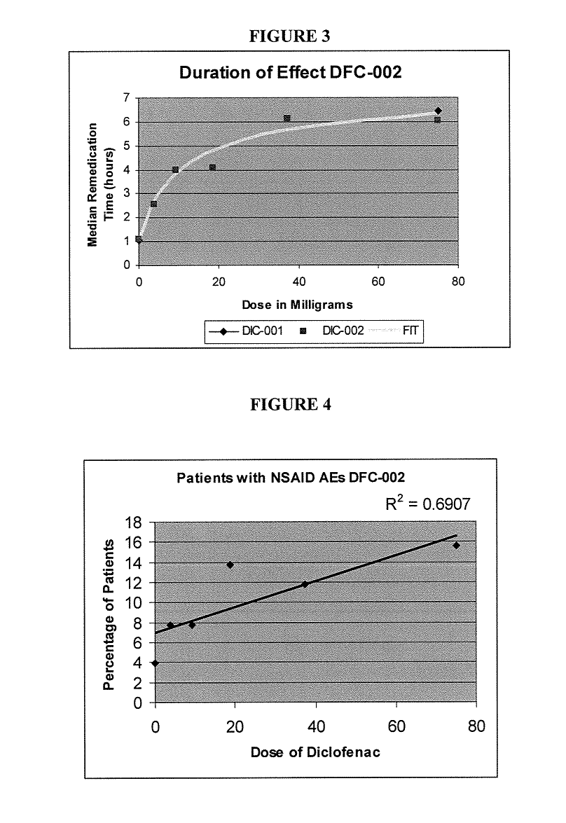

FIG. 3 depicts the dose-duration relationship examined using the median time to remedication in the single dose phase. The peak analgesic response was about 80% pain relief, with a 50% response at a dose of 4-8 milligrams of Diclofenac in relation to dental pain. Similar peak analgesic response was seen for 30 milligrams of ketorolac. Given the shape of the dose response curve, it is clear that lower doses of the DIC formulation achieved the same results as the current established dose of diclofenac of 75 milligrams.

The findings show a 6 hour duration of effect for all doses above about 20 milligrams (18 milligrams).

For most drugs the findings of significant activity at doses far below the recommended doses would be of little significance due to large therapeutic indices (wide ranges between the effective and toxic doses). The opposite is true for parenteral NSAIDs; it has been well established in the prior art that increasing the dose of these drugs rapidly diminishes their utility due to increasing risk of toxicity.

Thus the finding that with the new formulation of diclofenac that 5%-12% of the usual dose can provide 38-68% of the desired therapeutic effect is remarkable and unanticipated. This leads to the possibility that the high early blood levels possible with the new formulation allow lower total daily doses resulting in less risk of toxicity.

This finding demonstrates efficacy with a lower daily dose (25-75 mg/day) than current dosing of diclofenac (75-200 mg/day), and anticipates better dosing paradigms (less than Q 12 hours) offering the expectation of lesser toxicity. Proof of lesser toxicity from available data from this study is suggestive, based on the known relationship of dose and toxicity.

The novel diclofenac formulation allowed by hydroxypropyl-beta-cyclodextrin has been shown to provide proof of single-dose efficacy at 50%, 25%, 12.5% and 5% of the current recommended doses of diclofenac. This in combination with the known human pharmacokinetic results for the formulation supports reduced total daily doses of this NSAID with anticipated lower risk of toxicity by reducing the extent and duration of drug exposure.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further understood that all values are approximate, and are provided for description.

Patents, patent applications publications product descriptions, and protocols are cited throughout this application the disclosures of which are incorporated herein by reference in their entireties for all purposes.

What is claimed is:

1. A pharmaceutical composition comprising:
(a) a dosage of a non-steroidal anti-inflammatory drug (NSAID) effective to induce analgesia an anti-inflammatory effect, or an anti-pyretic effect and (b) a beta-cyclodextrin compound;
wherein the dosage of the NSAID compound is less than the minimum approved dose for the route of administration.

2. The pharmaceutical composition of claim 1, wherein the dosage of the NSAID is less than about 50% of the minimum approved dose for the route of administration.

3. The pharmaceutical composition of claim 2, wherein the dosage of the NSAID is less than about 25% of the minimum approved dose for the route of administration.

4. The pharmaceutical composition of claim 1, wherein the NSAID is diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib or lumaricoxib.

5. The pharmaceutical composition of claim 1, wherein the cyclodextrin compound is 2-hydroxypropyl-beta-cyclodextrin.

6. A method for treating a mammal in need of an analgesic, an anti-inflammatory, or an anti-pyretic agent comprising administering the pharmaceutical composition of claim 1.

7. The method for treating a mammal according to claim 6, wherein the pharmaceutical composition is administered intramuscularly.

8. The method for treating a mammal according to claim 6, wherein the pharmaceutical composition is administered intravenously.

9. A method for treating a mammal in need of an analgesic, an anti-inflammatory, or an anti-pyretic agent, which comprises administering a pharmaceutical composition comprising:
(a) a dosage of an NSAID effective to induce analgesia an anti-inflammatory effect, or an anti-pyretic effect; and
(b) a beta-cyclodextrin compound;
wherein the dosage of the NSAID is less than the minimum approved dose for the route of administration.

10. The method of claim 9, wherein the NSAID is diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib or lumaricoxib.

11. The method of claim 9, wherein the dose of the NSAID has the same efficacy as the minimum approved dosage.

12. The method of claim 9, wherein the dose of the NSAID has from about 70% to about 100% of the efficacy of the minimum approved dosage.

13. The method of claim 9, wherein the dosage of the NSAID has from about 40% to about 70% of the efficacy of the minimum approved dosage.

14. The method of claim 9, wherein the dosage of the NSAID has the same duration as the minimum approved dosage.

15. The method of claim 9, wherein the dosage of the NSAID has from about two-thirds to the same duration as the minimum approved dosage.

16. The method of claim 9, wherein the dosage of the NSAID has about one-third to about two-thirds of the duration as the minimum approved dosage.
